(19) United States
(12) Patent Application Publication
(10) Pub. No.: US 2011/0137065 A1
(43) Pub. Date: Jun. 9, 2011

(54) PROCESSES FOR SYNTHESIZING 1-(2-ETHYLBUTYL)CYCLOHEXANE CARBONITRILE

(76) Inventors: Christophe Durvaux, Bruxelles (BE); Thierry Giard, Mont Saint Guibert (BE); Vincent L. Mutterer, Bousval (BE)

(21) Appl. No.: 13/057,342

(22) PCT Filed: Apr. 28, 2009
(86) PCT No.: PCT/US2009/041877

§ 371 Date: Feb. 3, 2011

Related U.S. Application Data
(60) Provisional application No. 61/051,479, filed on May 8, 2008.

(51) Int. Cl. C07C 255/49

(52) U.S. Cl. 558/431

(57) ABSTRACT

Processes are provided for synthesizing substituted cyclohexane carbonitriles, particularly 1-(2-ethylbutyl)cyclohexane carbonitrile. Such processes comprise deprotonizing cyclohexane carbonitrile with chloro magnesium N,N-diisopropylamide and alkylating the deprotonated cyclohexane carbonitrile in the presence of 2-(ethylbutyl) bromide to produce 1-(2-ethylbutyl)cyclohexane carbonitrile.

### BACKGROUND

Substituted cyclohexane carbonitriles, such as 1-(2-ethylbutyl) cyclohexane carbonitrile, have considerable utility as intermediates in the pharmaceutical industry.

Currently available processes for synthesizing substituted cyclohexane carbonitriles, particularly 1-(2-ethylbutyl) cyclohexane carbonitrile, are performed in cryogenic conditions (−50° C. to −30° C.) in 62% yield (see, e.g., Tilford, C. H., Doerle, L. A., Van Campen Jr, M. G., Shelton M. G. J. Am. Chem. Soc 1949, 71, 1705). Metalated nitriles are typically generated by deprotonation with metal amide (M=Na, Li) (see, e.g., Fleming, F. F., Shook, B. C. Tetrahedron 2002, 58, 1. (b): Arsenyadis, S., Kyler, K. S., Watt D. S. Org. react. 1984, 31, 1). Such processes involve the formation of metal amide (when sodium amide is used), then the deprotonation of nitrile and subsequently the alkylation. The formation of sodium amide is performed from the reaction between sodium and ammonia and appropriate equipment must be provided to condense ammonia and to cool the reaction mixture during each step of the process.

It would be desirable to have new processes for synthesizing substituted cyclohexane carbonitriles, particularly 1-(2-ethylbutyl) cyclohexane carbonitrile, which do not require processing at temperatures below 0° C. Additionally, such processes that provide better yield than current processes would be advantageous.

### THE INVENTION

This invention meets the above-described needs by providing processes for synthesizing substituted cyclohexane carbonitriles, particularly 1-(2-ethylbutyl)cyclohexane carbonitrile. According to this invention, 1-(2-ethylbutyl) cyclohexane carbonitrile can be prepared from cyclohexane carbonitrile. After deprotonation with chloro magnesium N,N-diisopropylamide at about 0° C., the generated anion is alkylated in the presence of 2-(ethylbutyl) bromide. Typically, at least about a 92% yield is obtained.

Processes according to this invention comprise deprotonizing cyclohexane carbonitrile with chloro magnesium N,N-diisopropylamide and alkylating the deprotonated cyclohexane carbonitrile in the presence of 2-(ethylbutyl) bromide to produce 1-(2-ethylbutyl)cyclohexane carbonitrile.

### EXAMPLE

The following example is illustrative of the principles of this invention. It is understood that this invention is not limited to any one specific embodiment exemplified herein, whether in the examples or the remainder of this patent application.

### Example 1

Properties of reactants used in the Example, and of the product produced, are shown in Table 1:

TABLE 1

| | | | | | | | Mₚ | Bp | |
|---|---|---|---|---|---|---|---|---|---|
| Step | Chemical Name | CAS | MW | m/V | | mol | (° C.) | (° C.) | Density |
| 5 | Cyclohexane | 766-05-2 | 109.19 | 15 | g | 0.137 | 11 | 184 | 0.919 |
| | carbonitrile | | | | | | | | |
| 1 | N,N- | 108-18-9 | 101.19 | 15.36 | g | 0.152 | −61 | 84 | 0.722 |
| | Diisopropylamine | | | | | | | | |
| 3 | 2M Butyl | 693-04-9 | 116.87 | 76 | ml | 0.152 | — | — | 0.962 |
| | magnesium | | | | | | | | |
| | chloride in THF | | | | | | | | |
| 7 | 2-(Ethylbutyl) | 855425-38-6 | 165.07 | 26.48 | g | 0.137 | — | 142 | 1.179 |
| | bromide | | | | | | | | |
| 3 | Tetrahydrofurane | 109-99-9 | 72.11 | 13.2 | g | 0.18 | −108.4 | 66 | 0.88 |
| | (THF) | | | | | | | | |
| 1, 5, 7 | Toluene | 108-88-8 | 92.14 | 47.63 | g | 0.61 | −95 | 111 | 0.866 |
| 9 | Acetic acid | 64-19-7 | 60.04 | 9.86 | g | 0.164 | 16 | 117 | 1.048 |
| 14 | 1M K₂CO₃ | 584-08-7 | 138.21 | 4.7 | | 0.034 | 891 | — | — |
| 9, 10, | Water | 7732-18-5 | 18 | 240 | g | 13.33 | 0 | 100 | 1 |

| | | | | | | | |
|---|---|---|---|---|---|---|---|
| 12, 16 | | | | | | | |
| 19 | 1-(2-Ethylbutyl) | 855425-38-6 | 193.33 | 24.6 | 0.127 | 289 | 0.88 |
| | cyclohexane | | | | | | |
| | carbonitrile | | | | | | |
| | (product) | | | | | | |

This Example comprises the following steps:

1. At room temperature, N,N-diisopropylamine (15.36 g), toluene (15 ml) and tetrahydrofurane (THF) (15 ml) were combined in a three round bottom flask.
2. The combination of N,N-diisopropylamine and toluene (the “First Combination”) was cooled down to 0° C.
3. 2 M butyl magnesium chloride in THF and the First Combination were combined between 0 to 5° C.
4. The combination of the First Combination, butyl magnesium chloride, and THF (the “Second Combination”) was stirred for 45 min between 0 to 5° C.
5. Cyclohexane carbonitrile (15 g) diluted in toluene (20 ml) and the Second Combination were combined between 0 to 5° C.
6. The combination of the Second Combination, cyclohexane carbonitrile, and toluene (the “Third Combination”) was stirred between 0 to 5° C. for 1 hour.
7. 2-(Ethylbutyl) bromide (26.48 g) diluted in toluene (20 ml) and the Third Combination were combined between 0 to 10° C.
8. The combination of the Third Combination, 2-(ethylbutyl) bromide, and toluene (the “Fourth Combination”) was stirred for 4 h between 0 to 10° C.
9. Acetic acid (9.86 g) diluted in water (90 ml) was added to the Fourth Combination between 0 to 20° C.
10. Water (30 ml) was added to the combination of acetic acid diluted in water and Fourth Combination.
11. The combination of water, acetic acid diluted in water, and Fourth Combination (the “Fifth Combination”) was stirred for 15 min; thereafter an organic phase cut was made—i.e., the Fifth Combination was transferred into a separating funnel with a plug, where it separated into two layers, the aqueous layer (down) and the organic layer (up). The aqueous layer, which contained mainly magnesium salts and diisopropylammonium acetate, was removed and disposed of. The organic layer, containing the desired compound was left in the separating funnel.
12. Water (30 ml) and the extracted organic layer were combined.
13. The combination of water and the extracted organic layer (the “Sixth Combination”) were stirred for 15 min; thereafter a second organic phase cut was made.
14. 1 M K₂CO₃ (34.5 ml) and the extracted second organic layer were combined.
15. The combination of K₂CO₃ and the extracted second organic layer (the “Seventh Combination”) were stirred for 15 min; thereafter a third organic phase cut was made primarily to remove substantially all acidic traces.
16. Water (30 ml) and the extracted third organic layer were combined.
17. The combination of water and the extracted third organic layer (the “Eighth Combination”) was stirred for 15 min; thereafter a fourth organic phase cut was made primarily to remove substantially all carbonate traces.
18. The extracted fourth organic layer was distilled at 40° C. under 50 mbars to leave a bottoms; distilled solvents were disposed.
19. The bottoms was distilled at 170° C. under 5 mbars to produce 1-(2-ethyl butyl) cyclohexane carbonitrile

In this example, the addition of 2M butyl magnesium chloride to the N,N-diisopropylamine/toluene mixture (step 3) led to the deprotonation of N,N-diisopropylamine, which gave in-situ chloro magnesium diisopropyl amide (a strong base). Subsequently, when cyclohexane carbonitrile was added (step 5), there was a deprotonation performed by chloro magnesium diisopropyl amide. Then, when 2-(ethyl butyl) bromide (diluted in toluene) was added on metalated nitrile (step 7), the alkylation took place to generate 1-(2-ethylbutyl) cyclohexane carbonitrile. In step 19, 24.6 g of the desired product, 1-(2-ethylbutyl) cyclohexane carbonitrile, were produced, providing a yield of 92%.

In the practice of this invention, the Grignard reagent, e.g., butyl magnesium chloride or, alternatively, hexyl magnesium chloride, is transferred in inert condition. The addition of the Grignard reagent is exothermic. Formation of chloro magnesium diisopropylamide is exothermic. Addition of 2-(ethylbutyl) bromide is slightly exothermic. If desired, the Grignard reagent can be added to the flask or reactor and then a toluene mixture of diisopropylamine can be added.

Processes of this invention for synthesizing substituted cyclohexane carbonitriles are advantageous in that set up and around 0° C. is easier than set up at cryogenic temperatures. Additionally, processes of this invention allows the formation of the desired compound in at least about a 92% yield instead of 62% yield.

It is to be understood that the reactants and components referred to by chemical name or formula anywhere in the specification or claims hereof, whether referred to in the singular or plural, are identified as they exist prior to being combined with or coming into contact with another substance referred to by chemical name or chemical type (e.g., another reactant, a solvent, or etc.). It matters not what chemical changes, transformations and/or reactions, if any, take place in the resulting combination or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical reaction or in forming a combination to be used in conducting a desired reaction. Accordingly, even though the claims herein after may refer to substances, components and/or ingredients in the present tense (“comprises”, “is”, etc.), the reference is to the substance, component or ingredient as it existed at the time just before it was first contacted, combined, blended or mixed with one or more other substances, components and/or ingredients in accordance with the present disclosure. Whatever transformations, if any, which occur in situ as a reaction is conducted is what the claim is intended to cover. Thus the fact that a substance, component or ingredient may have lost its original identity through a chemical reaction or transformation during the course of contacting, combining, blending or mixing operations, if conducted in accordance with this disclosure and with the application of common sense and the ordinary skill of a chemist, is thus wholly immaterial for an accurate understanding and appreciation of the true meaning and substance of this disclosure and the claims thereof. As will be familiar to those skilled in the art, the terms “combined”, “combining”, and the like as used herein mean that the components that are “combined” or that one is “combining” are put into a container with each other. Likewise a “combination” of components means the components having been put together in a container.

While the present invention has been described in terms of one or more preferred embodiments, it is to be understood that other modifications may be made without departing from the scope of the invention, which is set forth in the claims below.

What is claimed is:

1. A process comprising:
deprotonizing cyclohexane carbonitrile with chloro magnesium N,N-diisopropylamide;
alkylating the deprotonated cyclohexane carbonitrile in the presence of 2-(ethylbutyl) bromide; and
producing 1-(2-ethylbutyl)cyclohexane carbonitrile.

2. The process of claim 1 wherein deprotonizing cyclohexane carbonitrile with chloro magnesium N,N-diisopropylamide comprises:
combining N,N-diisopropylamine and toluene to form a First Combination;
producing chloro magnesium N,N-diisopropylamide;
combining 2M butyl magnesium chloride, additional tetrahydrofurane, produced chloro magnesium N,N-diisopropylamide and the First Combination to form a Second Combination; and
combining cyclohexane carbonitrile, toluene, and the Second Combination to form a Third Combination.

3. The process of claim 2 wherein alkylating the deprotonated cyclohexane carbonitrile in the presence of 2-(ethylbutyl) bromide comprises:
combining 2-(ethylbutyl) bromide and the Third Combination to form a Fourth Combination; and
combining water, acetic acid, and the Fourth Combination to form a Fifth Combination.

4. The process of claim 3 wherein the Fifth Combination comprises an organic phase and producing 1-(2-ethylbutyl) cyclohexane carbonitrile comprises distilling 1-(2-ethylbutyl) cyclohexane carbonitrile from at least a portion of the organic phase.
